(19) United States
(12) Patent Application Publication
(10) Pub. No.: US 2013/0078238 A1
(43) Pub. Date: Mar. 28, 2013

(54) Methods and Compositions for Treating Hepatitis with Anti-CD3 Immune Molecule Therapy

(71) Applicants: Yaron Ilan, Jerusalem (IL); Ronald Ellis, Jerusalem (IL)

(72) Inventors: Yaron Ilan, Jerusalem (IL); Ronald Ellis, Jerusalem (IL)

(73) Assignees: Hadasit Medical Research Services & Decvelopment Co. Ltd., Jerusalem (IL); NASVAX LTD., Ness-Ziona (IL)

(21) Appl. No.: 13/635,119

(22) PCT Filed: Apr. 29, 2011
(86) PCT No.: PCT/IB11/51900

§ 371 Date: Dec. 10, 2012

Related U.S. Application Data
(60) Provisional application No. 61/329,554, filed on Apr. 29, 2010.

(51) Int. Cl. A61K 39/395

(52) U.S. Cl. CPC A61K 39/3955
USPC 424/133.1; 424/173.1; 424/144.1; 424/142.1; 530/389.6; 530/388.22; 530/387.3; 530/388.15

(57) ABSTRACT

A method or composition comprising an anti-CD3 immune molecule for treatment of hepatitis in a subject.

### FIELD OF THE INVENTION

The present invention relates to methods and compositions for treating hepatitis, and in particular, for treating hepatitis with anti-CD3 immune molecules, such as antibodies, administered orally or mucosally.

### BACKGROUND OF THE INVENTION

Immunotherapy strategies that involve antibody-induced signaling through antigen-specific T-cell receptors (TCR) have been shown to ameliorate autoimmune and inflammatory diseases, probably by regulating the immune response to self-antigens. One example of such a receptor is CD3 (cluster of differentiation 3). Parenterally administered anti-CD3 monoclonal antibody (mAb) therapy in particular has been shown to be efficacious in preventing and reversing the onset of diabetes in NOD mice (Chatenoud et al., J. Immunol. 158:2947-2954 (1997); Belghith et al., Nat. Med. 9:1202-1208 (2003)) and in treating subjects with Type 1 diabetes (Herold et al., N. Engl. J. Med. 346 (22):1692-1698 (2002)), and to reverse experimental allergic encephalomyelitis (EAE) in Lewis rats with a preferential suppressive effect on T-helper type 1 (Th1) cells, which participate in cell-mediated immunity (Tran et al., Intl. Immunol. 13 (9):1109-1120 (2001)). The FDA approved Orthoclone OKT3 (muromonab-CD3; Ortho Biotech Products, Bridgewater, N.J.), a murine anti-CD3 mAb, for intravenous injection for the treatment of graft rejection after transplantation (Chatenoud, Nat. Rev. Immunol. 3:123-132 (2003)).

As described in U.S. Pat. No. 7,883,703 to Howard Weiner et al., which is hereby incorporated by reference as if fully set forth herein, anti-CD3 antibodies are also useful for treatment of autoimmune diseases when administered orally or mucosally. Without wishing to be limited by a single hypothesis, the success of such oral or mucosal administration is attributed to activation of regulatory T cells (Treg) in the mucosal immune system, which in turn leads to an amelioration or down-regulation of the undesired immune system effects, hence ameliorating or at least reducing the pathology of the autoimmune and inflammatory disease. Among the advantages of the oral or mucosal route over the systemic route of administration of anti-CD3 mAb is the ability to avoid the serious adverse events and generalized immune-suppression associated with systemic administration. This route of administration also acts to increase regulatory T cells and to suppress effector cells thus alleviating inflammatory disorders.

### SUMMARY OF THE INVENTION

The background art does not teach or suggest methods or compositions for treatment of hepatitis with anti-CD3 oral or mucosal immune molecule therapy.

The present invention, in at least some embodiments, overcomes the limitations of the background art by providing methods and compositions for treatment of hepatitis with anti-CD3 oral or mucosal immune molecule therapy. As used herein, the term “treatment” of hepatitis also encompasses preventing progression and/or delaying development of hepatitis.

“Oral or mucosal immune molecule therapy” means the administration of an active anti-CD3 immune molecule orally or to a mucosal membrane (or a combination thereof). Such an anti-CD3 immune molecule may optionally and preferably comprise an anti-CD3 antibody, for example and without limitation, whole antibodies or active fragments thereof (e. g., F (ab′)₂ or scFv, etc). For the purpose of description only and without wishing to be limited in any way, reference may be made herein to an anti-CD3 antibody; it is understood that such a reference may refer to any anti-CD3 immune molecule that is suitable for oral or mucosal administration.

The term “hepatitis” refers to any inflammation of the liver. Non-limiting examples of causes of hepatitis include infectious agents (including but not limited to viruses such as hepatitis A, B, C, D and E; herpes viruses such as herpes simplex virus (HSV); cytomegalovirus; Epstein-Barr virus; and other viruses such as yellow fever virus, HIV (human immunodeficiency virus), and adenoviruses; and non-viral infectious agents such as toxoplasma, Leptospira, Q fever and Rocky Mountain spotted fever; or any infectious agent resulting in hepatitis); toxins (including any toxic substance or any substance which is toxic to the liver with excessive intake, such as alcohol or medicines, for example due to any drug associated liver injury (DILI), including acetaminophen or any other drug that leads to liver damage); non-alcoholic steatohepatitis, or NASH (non-alcoholic steato-hepatitis), which is caused or exacerbated by obesity or diabetes, or a combination of these two conditions; liver disease associated with inflammatory bowel disease; hyperlipidemia, whether as the primary or only cause, or in association with NASH; and as a consequence of vascular disorders; or hepatitis of other etiology.

According to at least some embodiments of the present invention, there is provided a method for treating hepatitis by administering an anti-CD3 immune molecule, such as an anti-CD3 antibody, orally or mucosally, for example and without limitation, via pulmonary, buccal, nasal, intranasal, sublingual, rectal, or vaginal administration. The hepatitis may optionally be caused by any factor or combinations of factors, such as those described herein.

Optionally and preferably, the hepatitis is caused by the virus hepatitis C (HCV) or by NASH, such that in these embodiments, the method features administering an anti-CD3 immune molecule orally or mucosally to treat hepatitis caused by HCV or by NASH.

In at least some embodiments, there are provided pharmaceutical compositions for treatment of hepatitis suitable for oral or mucosal administration including an anti-CD3 antibody (or any other anti-CD3 immune molecule). In some embodiments, the pharmaceutical composition is suitable for pulmonary, buccal, nasal, intranasal, sublingual, rectal, or vaginal administration. In some embodiments, the anti-CD3 antibody is selected from the group consisting of a murine mAb, a humanized mAb, a human mAb, and a chimeric mAb. In some embodiments, the composition suitable for oral administration is in a form selected from a liquid oral dosage form and a solid oral dosage form, e. g., selected from the group consisting of tablets, capsules, caplets, powders, pellets, granules, powder in a sachet, enteric coated tablets, enteric coated beads, encapsulated powders, encapsulated pellets, encapsulated granules, and enteric coated soft gel capsules. In some embodiments, the oral dosage form is a controlled release oral formulation.

In some embodiments, the pharmaceutical compositions further comprise excipients and/or carriers. In some embodiments, the pharmaceutical compositions further comprise additional active or inactive ingredients.

In an additional aspect, the invention provides methods of providing an anti-CD3 antibody to a subject for treatment of hepatitis. The methods for treatment of hepatitis can include administering to the subject an oral dosage form suitable to deliver a dosage of an anti-CD3 antibody via the gastrointestinal tract, which, upon oral administration, leads to amelioration of hepatitis and inflammation.

In a further aspect, the invention provides methods of providing an anti-CD3 antibody to a subject for treatment of hepatitis. The methods include administering to the subject an oral dosage form suitable to deliver a dosage of an anti-CD3 antibody via the gastrointestinal tract, which, without wishing to be limited by a single hypothesis, upon oral administration, leads to stimulating the development of Treg cells with resultant amelioration in hepatitis.

Alternatively, the methods for treatment of hepatitis can include administering to the subject a mucosal dosage form suitable to deliver a dosage of an anti-CD3 antibody via a mucous membrane, which, upon mucosal administration and again without wishing to be limited by a single hypothesis, leads to stimulating the development of Treg cells with resultant amelioration in hepatitis.

The invention, in at least some embodiments, provides several advantages, in addition to its efficacy for treatment of hepatitis, such as hepatitis caused by a virus such as HCV and/or hepatitis caused by NASH and/or hepatitis arising from other causes. Without wishing to be limited to a closed list, these advantages over known methods of treatment include the following. First, oral or mucosal administration is easier to accomplish and is generally preferred over parenteral administration (e.g., intravenous or by injection) by the majority of subjects, due to the lack of needles and needlesticks associated with chronic therapy, hence improved compliance by subjects. Second, oral or mucosal administration facilitates chronic administration of the antibody. Third, oral or mucosal administration can avoid or reduce the negative side effects and pain associated with parenteral administration, including injection site pain. Fourth, oral or mucosal administration can avoid the serious side effects associated with parenteral administration of antibody, including generalized immunosuppression and cytokine storm. Other advantages include but are not limited to reduced costs, since highly trained personnel are not required for oral or mucosal administration, and fewer safety concerns for both subjects and medical staff that are using sharp needles. In some circumstances but without wishing to be limited by a closed list, orally or mucosally administered anti-CD3 antibodies result in reduced inflammation and/or auto-immune disease at a lower dosage than parenterally administered anti-CD3 antibodies and without the side effects of parenteral administration.

Moreover, oral or mucosal antibodies can be effective when administered both before development of the disease and during the ascending period of disease and when given at the peak of the disease, while parenterally administered antibodies are commonly believed to be effective only after onset of the disease (Chatenoud et al., J. Immunol. 158: 2947-2954 (1997); Tran et al., Int. Immunol. 13: 1109-1120 (2001)).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 relates to mean blood glucose levels;

FIG. 2 relates to the mean of changes for the AUC (area under the curve) for a glucose tolerance test;

FIG. 3 relates to AST levels;

FIG. 4 relates to the percentage of change in the CD4⁺LAP⁺ population; and

FIG. 5 relates to the percentage of change in TGFβ levels.

### DETAILED DESCRIPTION

The present invention, in at least some embodiments, provides methods of treating hepatitis via oral or mucosal administration of anti-CD3 antibodies and compositions suitable for oral or mucosal administration of anti-CD3 antibodies.

Hepatitis, as noted above, refers to any inflammation of the liver. A more detailed description is provided herein of two non-limiting examples for treating causes of hepatitis, NASH and HCV.

NASH is characterized by fat in the liver, along with inflammation and damage, which is not due to excessive amounts of alcohol ingestion. Nevertheless, NASH can be severe and can lead to cirrhosis of the liver and even liver fibrosis. It is caused by obesity and diabetes, and is potentiated in patients suffering from both conditions. However, NASH may also occur in patients without either condition. Without wishing to be limited by a single hypothesis, NASH also may be caused by insulin resistance, release of toxic inflammatory proteins by fat cells (cytokines) and/or oxidative stress (deterioration of cells) inside liver cells. Currently, there are no effective treatments for NASH.

Hepatitis C virus (HCV) is one type of hepatitis virus (the others including A, B, D and E). HCV is a small (40-60 nanometers in diameter), enveloped, single-stranded RNA virus of the family Flaviviridae and genus hepacivirus. Because the virus mutates rapidly, changes in the envelope proteins may help it evade the immune system. There are at least six major genotypes and more than 50 subtypes of HCV.

HCV is one of the most important causes of chronic liver disease in the United States. It accounts for about 15 percent of acute viral hepatitis, 60-70 percent of chronic hepatitis, and up to 50 percent of cirrhosis, end-stage liver disease, and liver cancer. These latter acute aspects of the disease are caused by chronic hepatitis C following acute HCV infection, which can cause cirrhosis (leading to fibrosis), liver failure, and liver cancer.

As described herein, hepatitis may be treated through oral or mucosal administration of an anti-CD3 immune molecule therapy. The usefulness of an oral formulation requires that the active agent be bioavailable. Bioavailability of orally administered drugs can be affected by a number of factors, such as drug absorption throughout the gastrointestinal tract, stability of the drug in the gastrointestinal tract, and the first pass effect. Thus, effective oral delivery of an active agent requires that the active agent have sufficient stability during traversal of the stomach and intestinal lumen to reach and pass through the intestinal wall to the lamina propria. Many drugs, however, tend to degrade quickly in the intestinal tract or have poor absorption in the intestinal tract so that oral administration is not an effective method for administering the drug. Surprisingly, not only can anti-CD3 antibodies be administered orally, but it appears that oral administration is, in some aspects, superior to parenteral administration in terms of positive immune-modulatory activity and in a practical way.

Within the immune system, a series of anatomically distinct compartments can be distinguished, each specially adapted to respond to pathogens present in a particular set of body tissues. One compartment, the peripheral compartment, comprises the peripheral lymph nodes and spleen; this compartment responds to antigens that enter the tissues or spread into the blood. A second compartment, the mucosal immune system, is located near the mucosal surfaces where most pathogens invade. The mucosal immune system has evolved antigen-specific tolerance mechanisms to avoid a deleterious immune response to food antigens and beneficial, commensal microorganisms, which live in symbiosis with their host, while still detecting and killing pathogenic organisms that enter through the gut. Generally speaking, the gut-associated lymphoid tissue (GALT) is different from lymphoid tissue elsewhere; stimulation of the GALT preferentially induces regulatory T cells (Treg). Anti-CD3 immune molecules (such as anti-CD3 antibodies) are rapidly taken up by the gut associated-lymphoid tissue and induce CD4+CD25-LAP+ Tregs. The cells from the gut lymphoid tissue secrete mainly TGF-β and IL-10, and the chance and the frequency of stimulating regulatory cells is higher in the gut.

Immune responses induced within one compartment are largely confined to that particular compartment. Lymphocytes are restricted to particular compartments by their expression of homing receptors that are bound by ligands, known as addressins, which are specifically expressed within the tissues of the compartment. Interestingly, tolerance induced in the mucosal compartment also applies and transfers to the peripheral compartment. For example, the feeding of ovalbumin (a strong parenteral antigen) is followed by an extended period during which the administration of ovalbumin by injection, even in the presence of adjuvant, elicits no antibody response in either the peripheral compartment or the mucosal compartment. In contrast, oral tolerance is a systemic tolerance; although the induction of oral tolerance occurs in the gut, peripheral tolerance also results.

Without wishing to be limited by a single hypothesis, orally administered anti-CD3 immune molecules stimulate the mucosal immune system. As noted above, the gut is a unique environment in which to induce tolerance. In comparison with parenterally administered antibodies, lower amounts of oral anti-CD3 antibodies are needed to induce tolerance and do so without stimulating general immune-suppression and other serious side effects; in addition, oral antibodies can be effective when administered both before and during the development of the disease and when given at the peak of the disease, while parenterally administered antibodies are effective only after onset of the disease.

Pharmaceutical Compositions

Pharmaceutical compositions suitable for oral administration are typically solid dosage forms (e. g., tablets) or liquid preparations (e.g., solutions, suspensions, emulsions, or elixirs).

Solid dosage forms are desirable for ease of determining and administering defined dosage of active ingredient, and ease of administration, particularly administration by the subject at home.

Liquid dosage forms also allow subjects to easily take the required dose of active ingredient; liquid preparations can be prepared as a drink, or to be administered, for example, by a naso- gastric tube.

Liquid oral pharmaceutical compositions generally require a suitable solvent or carrier system in which to dissolve or disperse the active agent, thus enabling the composition to be administered to a subject. A suitable solvent system is compatible with the active agent and non-toxic to the subject. Typically, liquid oral formulations use a water-based solvent.

The oral compositions can also optionally be formulated to reduce or avoid the degradation, decomposition, or deactivation of the active agent by the gastrointestinal system, e.g., by gastric fluid in the stomach. For example, the compositions can optionally be formulated to pass through the stomach unaltered and to dissolve in the intestines, i.e., as enteric coated compositions.

One of ordinary skill in the art would readily appreciate that the pharmaceutical compositions described herein can be prepared by applying known pharmaceutical manufacturing procedures as established through a long history of application for oral products. Such formulations can be administered to the subject with methods well-known in the pharmaceutical arts. Thus, the practice of the present methods will employ, unless otherwise indicated, conventional techniques of pharmaceutical sciences including pharmaceutical dosage form design, drug development, and pharmacology, as well as of organic chemistry, including polymer chemistry. Accordingly, these techniques are within the capabilities of one of ordinary skill in the art and are explained fully in the literature (See generally, for example, Remington: The Science and Practice of Pharmacy, Nineteenth Edition. Alfonso R. Gennaro (Ed.): Mack Publishing Co., Easton, Pa., (1995), hereinafter Remington, incorporated by reference herein in its entirety).

Anti-CD3 Immune Molecules

An anti-CD3 immune molecule may for example optionally comprise any anti-CD3 antibody. The anti-CD3 antibodies can be any antibodies specific for CD3. The term “antibody” as used herein refers to an immunoglobulin molecule or immunologically active portion thereof that is readily derived by means of known techniques of protein chemistry and recombinant DNA engineering, i.e., an antigen-binding portion. Non-limiting examples of immunologically active portions of immunoglobulin molecules include F(ab) and F(ab′)₂ fragments, which retain the ability to bind CD3. Such fragments can be obtained commercially or by using methods known in the art. For example F(ab)₂ fragments can be generated by treating the antibody with an enzyme such as pepsin, a non-specific endopeptidase that normally produces one F(ab)₂ fragment and numerous small peptides of the Fc portion. The resulting F(ab)₂ fragment is composed of two disulfide-connected Fab units. The Fc fragment is extensively degraded and can be separated from the F(ab)₂ by dialysis, gel filtration or ion exchange chromatography. F(ab) fragments can be generated using papain, a non-specific thiol-endopeptidase that digests IgG molecules, in the presence of a reducing agent, into three fragments of similar size: two Fab fragments and one Fc fragment. When Fc fragments are of interest, papain is the enzyme of choice because it yields a 50,000 Dalton Fc fragment; to isolate the F (ab) fragments, the Fc fragments can be removed, e.g., by affinity purification using protein A/G. A number of kits are available commercially for generating F(ab) fragments, including the ImmunoPure IgG1 Fab and F(ab′)2 Preparation Kit (Pierce Biotechnology, Rockford, Ill.). In addition, commercially available services for generating antigen-binding fragments can be used, e g., Bio Express, West Lebanon, N.H.

The antibody may optionally be a polyclonal, monoclonal, recombinant, e.g., a chimeric, de-immunized or humanized, fully human, non-human, e.g., murine, or single chain antibody.

In some embodiments the antibody has effector function and can fix complement. In some embodiments, the antibody has reduced or no ability to bind an Fc receptor. For example, the anti-CD3 antibody can be an isotype or subtype, fragment or other mutant, which does not support binding to an Fc receptor, e. g., it has a mutagenized or deleted Fc receptor binding region. The antibody can be coupled to a toxin or imaging agent.

A number of anti-CD3 antibodies are known, including but not limited to OKT3 (muromonab/Orthoclone OKT3™, Ortho Biotech, Raritan, N.J.; U.S. Pat. No. 4,361,549); hOKT3Y1 (Herold et al., N. E. J. M. 346 (22): 1692-1698 (2002); HuM291 (Nuvion™, Protein Design Labs, Fremont, Calif.); gOKT3-5 (Alegre et al., J. Immunol. 148 (11): 3461-8 (1992); 1F4 (Tanaka et al., J. Immunol. 142: 2791-2795 (1989)); G4.18 (Nicolls et al., Transplantation 55: 459-468 (1993)) ; 145-2C11 (Davignon et al., J. Immunol. 141 (6): 1848-54 (1988)); and as described in Frenken et al., Transplantation 51 (4): 881-7 (1991); U.S. Pat. Nos. 6,491,9116, 6,406,696, and 6,143,297). However any suitable anti-CD3 antibody may be used with the methods and compositions of the present invention.

Methods for making such antibodies are also known. A full-length CD3 protein or antigenic peptide fragment of CD3 can be used as an immunogen, or can be used to identify anti-CD3 antibodies made with other immunogens, e. g., cells, membrane preparations, and the like, e. g., E rosette positive purified normal human peripheral T cells, as described in U.S. Pat. No. 4,361,549 and 4,654,210. The anti-CD3 antibody can bind an epitope on any domain or region on CD3 for retaining functionality.

Chimeric antibodies contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e. g., Cabilly et al., U.S. Pat. No. 4,816,567; Shoemaker et al., U.S. Pat. No. 4,978,745; Beavers et al., U.S. Pat. No. 4,975,369; and Boss et al., U. S. Pat. No. 4,816,397, all of which are incorporated by reference herein. Generally, these chimeric antibodies are constructed by preparing a genomic gene library from DNA extracted from pre-existing murine hybridomas (Nishimura et al. Cancer Research, 47: 999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e. g., a murine myeloma line. Such chimeric antibodies have been used in human therapy. Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81: 6801 (1984); Morrison and Oi, Adv. Immunol. 44: 65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321: 522 (1986); Verhoeyen et al., Science 239: 1539 (1988)) ; or (c) by transplanting the entire nonhuman variable domains (to preserve ligand- binding properties) but also “cloaking” them with a human-like surface through judicious replacement of exposed residues (to reduce antigenicity) (Padlan, Molec. Immunol. 28: 489 (1991)).

However, given use of the oral or mucosal delivery routes of the antibodies according to at least some embodiments of the present invention, such humanization or reduced immunogenicity may not be necessary.

The anti-CD3 antibody can also be a single chain antibody. A single-chain antibody (scFV) can be engineered (see, for example, Colcher et al., Ann N. Y. Acad. Sci. 880: 263-80 (1999); and Reiter, Clin. Cancer Res. 2: 245-52 (1996)). The single chain antibody can be dimerized or multimerized to generate multivalent antibodies having specificities for different epitopes of the same target CD3 protein. In some embodiments, the antibody is monovalent, e. g., as described in Abbs et al., Ther. Immunol. 1 (6): 325-31 (1994), incorporated herein by reference.

Pharmaceutical Compositions with Anti-CD3 Antibodies

The anti-CD3 antibodies described herein can be incorporated into a pharmaceutical composition suitable for oral or mucosal administration, e. g., by ingestion, inhalation, or absorption, e. g., via nasal, intranasal, pulmonary, buccal, sublingual, rectal, or vaginal administration. Such compositions can include an inert diluent or an edible carrier. For the purpose of oral therapeutic administration, the active compound (e. g., an anti-CD3 antibody) can be prepared with excipients and used in solid or liquid (including gel) form. Oral anti-CD3 antibody compositions can also be prepared using an excipient. Pharmaceutically compatible binding agents can be included as part of the composition. Oral dosage forms comprising anti-CD3 antibody are provided, wherein the dosage forms, upon oral administration, provide a therapeutically effective mucosal level of anti-CD3 antibody to a subject. Also provided are mucosal dosage forms comprising anti-CD3 antibody wherein the dosage forms, upon mucosal administration, provide a therapeutically effective mucosal level of anti-CD3 antibody to a subject. For the purpose of mucosal therapeutic administration, the active compound (e.g., an anti-CD3 antibody) can be incorporated with excipients or carriers suitable for administration by inhalation or absorption, e. g., via nasal sprays or drops, or rectal or vaginal suppositories.

Solid oral dosage forms include, but are not limited to, tablets (e. g. chewable tablets), capsules, caplets, powders, pellets, granules, powder in a sachet, enteric coated tablets, enteric coated beads, and enteric coated soft gel capsules. Also included are multi-layered tablets, wherein different layers can contain different drugs. Solid dosage forms also include powders, pellets and granules that are encapsulated. The powders, pellets, and granules can be coated, e. g., with a suitable polymer or a conventional coating material to achieve, for example, greater stability in the gastrointestinal tract, or to achieve a desired rate of release.

In addition, a capsule comprising the powder, pellets or granules can be further coated. A tablet or caplet can be scored to facilitate division for ease in adjusting dosage as needed.

The dosage forms of the present invention can be unit dosage forms wherein the dosage form is intended to deliver one therapeutic dose per administration, e. g., one tablet is equal to one dose. Such dosage forms can be prepared by methods of pharmacy well known to those skilled in the art (see Remington's Pharmaceutical Sciences, 18th ed., Mack Publishing, Easton Pa. (1990)).

Typical oral dosage forms can be prepared by combining the active ingredients in an intimate admixture with at least one excipient according to conventional pharmaceutical compounding techniques. Excipients can take a wide variety of forms depending on the form of preparation desired for administration. For example, excipients suitable for use in solid oral dosage forms (e. g., powders, tablets, capsules, and caplets) include, but are not limited to, starches, sugars, micro-crystalline cellulose, diluents, granulating agents, lubricants, binders, and disintegrating agents. Examples of excipients suitable for use in oral liquid dosage forms include, but are not limited to, water, glycols, oils, alcohols, flavoring agents, preservatives, and coloring agents.

Tablets and capsules represent convenient pharmaceutical compositions and oral dosage forms, in which case solid excipients are employed. If desired, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

As one example, a tablet can be prepared by compression or by molding. Compressed tablets can be prepared, e. g., by compressing, in a suitable machine, the active ingredients (e. g., an anti-CD3 antibody) in a free-flowing form such as powder or granules, optionally mixed with an excipient. Molded tablets can be made, e. g., by molding, in a suitable machine, a mixture of the powdered anti-CD3 antibody compound moistened, e. g., with an inert liquid diluent.

Excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gum tragacanth or gelatin, natural and synthetic gums such as acacia, sodium alginate, alginic acid, other alginates, powdered tragacanth, guar gum, cellulose and its derivatives (e. g., ethyl cellulose, cellulose acetate, carboxymethyl cellulose calcium, sodium carboxymethyl cellulose), polyvinyl pyrrolidinones, methyl cellulose, pre-gelatinized starch, hydroxypropyl methyl cellulose, (e. g., Nos. 2208, 2906, 2910), microcrystalline cellulose, and mixtures thereof.

Suitable forms of microcrystalline cellulose include, but are not limited to, the materials sold as AVICEL PH-101, AVICEO PH-103 AVICEL RC-581, AVICEO PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEO RC-581. Suitable anhydrous or low moisture excipients or additives include AVICEL PH-103 and Starch 1500.

Examples of fillers suitable for use in the pharmaceutical compositions and dosage forms disclosed herein include, but are not limited to, talc, calcium carbonate (e. g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions and dosage forms of the invention is typically present in from about 50 to about 99 weight percent of the pharmaceutical composition or dosage form.

Disintegrants can be used in the pharmaceutical compositions and oral or mucosal dosage forms of the invention to provide tablets that disintegrate when exposed to an aqueous environment. Tablets containing too much disintegrant might disintegrate in storage, while those containing too little might not disintegrate at a desired rate or under desired conditions.

Thus, a sufficient amount of disintegrant that is neither too much nor too little to detrimentally alter the release of the active ingredients should be used to form the pharmaceutical compositions and solid oral dosage forms described herein. The amount of disintegrant used varies based upon the type of formulation, and is readily discernible to those of ordinary skill in the art. Typically, pharmaceutical compositions and dosage forms comprise from about 0.5 to about 15 weight percent of disintegrant, preferably from about 1 to about 5 weight percent of disintegrant.

Disintegrants that can be used in pharmaceutical compositions and oral or mucosal dosage forms of the invention include, but are not limited to, agar-agar, alginic acid, calcium carbonate, Primogel, microcrystalline cellulose, croscarmellose sodium, crospovidone, polacrilin potassium, sodium starch glycolat corn, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate or

Sterotes, mineral oil, light mineral oil, glycerin, sorbitol, mannitol, polyethylene glycol, other glycols, stearic acid, sodium lauryl sulfate, talc, hydrogenated vegetable oil (e. g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSILe 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated. A glidant such as colloidal silicon dioxide can also be used.

The pharmaceutical compositions and oral or mucosal dosage forms can further comprise one or more compounds that reduce the rate by which an active ingredient will decompose. Thus the oral dosage forms described herein can be processed into an immediate release or a sustained release dosage form Immediate release dosage forms may release the anti-CD3 antibody in a fairly short time, for example, within a few minutes to within a few hours. Sustained release dosage forms may release the anti-CD3 antibody over a period of several hours, for example, up to 24 hours or longer, if desired. In either case, the delivery can be controlled to be substantially at a certain predetermined rate over the period of delivery. In some embodiments, the solid oral dosage forms can be coated with a polymeric or other known coating material(s) to achieve, for example, greater stability on the shelf or in the gastrointestinal tract especially for traversing the stomach's acidic pH, or to achieve control over drug release. Such coating techniques and materials used therein are well-known in the art. Such compounds, which are referred to herein as “stabilizers”, include, but are not limited to, antioxidants such as ascorbic acid and salt buffers. For example, cellulose acetate phthalate, polyvinyl acetate phthalate, hydroxypropylmethyl cellulose phthalate, methacrylic acid-methacrylic acid ester copolymers, cellulose acetate trimellitate, carboxymethylethyl cellulose, and hydroxypropylmethyl cellulose acetate succinate, among others, can be used to achieve enteric coating. Mixtures of waxes, shellac, zein, ethyl cellulose, acrylic resins, cellulose acetate, silicone elastomers can be used to achieve sustained release coating. See, for example, Remington, supra, Chapter 93, for other types of coatings, techniques and equipment.

Liquids for oral or mucosal administration represent another convenient dosage form, in which case a solvent can be employed. In some embodiments, the solvent is a buffered liquid such as phosphate buffered saline (PBS). Liquid oral dosage forms can be prepared by combining the active ingredient in a suitable solvent to form a solution, suspension, syrup, emulsion, or elixir of the active ingredient in the liquid. The solutions, suspensions, syrups, emulsions and elixirs may optionally comprise other additives including, but not limited to, glycerin, sorbitol, propylene glycol, sugars or other sweeteners, flavoring agents, and stabilizers. Flavoring agents can include, but are not limited to peppermint, methyl salicylate, or orange flavoring.

Sweeteners can include sugars, aspartame, acesulfame-K, saccharin, sodium cyclamate and xylitol.

In order to reduce the degree of inactivation of orally administered anti-CD3 antibody in the stomach of the treated subject as a result of acidic pH, an antacid can be administered simultaneously with the immunoglobulin, which neutralizes the otherwise acidic character of the gut. Thus in some embodiments, the anti-CD3 antibody is administered orally with an antacid, e. g., aluminum hydroxide or magnesium hydroxide such as MAALOX antacid or MYLANTA antacid, or an H2 blocker, such as cimetidine or ranitidine, or proton pump inhibitor such as a member of the benzimidazole family, such as omeprazole. One of skill in the art will appreciate that the dose of antacid administered in conjunction with an anti-CD3 antibody depends on the particular antacid used. When the antacid is MYLANTA antacid in liquid form, between 15 ml and 30 ml can be administered, e. g., about 15 ml. When the cimetidine H2 blocker is used, between about 400 and 800 mg per day can be used. When the proton pump inhibitor is used, between about 10 and 40 mg per day can be used.

The kits described herein can include an oral anti-CD3 antibody composition as an already prepared liquid oral dosage form ready for administration or, alternatively, can include an anti-CD3 antibody composition as a solid pharmaceutical composition that can be reconstituted with a solvent to provide a liquid oral dosage form. When the kit includes an anti-CD3 antibody composition as a solid pharmaceutical composition that can be reconstituted with a solvent to provide a liquid dosage form (e. g., for oral or nasal administration), the kit may optionally include a reconstituting solvent. In this case, the constituting or reconstituting solvent is combined with the active ingredient to provide a liquid oral dosage form of the active ingredient. Typically, the active ingredient is soluble in the solvent and forms a solution. The solvent can be, e. g., water, a non-aqueous liquid, or a combination of a non-aqueous component and an aqueous component. Suitable non-aqueous components include, but are not limited to oils; alcohols, such as ethanol; glycerin; and glycols, such as polyethylene glycol and propylene glycol. In some embodiments, the solvent is PBS.

For administration by inhalation, the mucosal anti-CD3 antibody compounds can be delivered in the form of an aerosol spray from pressured container or dispenser which contains a suitable propellant, e g., a gas such as carbon dioxide, or a nebulizer. Such methods include those described in U.S. Pat. No. 6,468,798.

The anti-CD3 antibody compounds can also be prepared in the form of suppositories (e. g., with conventional suppository bases such as cocoa butter and other glycerides) or retention enemas for rectal or vaginal delivery, or for sprays for nasal or pulmonary delivery.

In one embodiment, the oral or mucosal anti-CD3 antibody compositions are prepared with carriers that will protect the anti-CD3 antibody against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U. S. Patent No. 4,522, 811.

Dosage, toxicity and therapeutic efficacy of such anti-CD3 antibody compositions can be determined by standard pharmaceutical procedures in cell cultures (e. g., of cells taken from an animal after mucosal administration of an anti-CD3 antibody) or experimental animals, e. g., for determining the LD₅₀ (the dose lethal to 50% of the study group) and the ED₅₀ (the dose therapeutically effective in 50% of the study group). The dose ratio between toxic and therapeutic effects is the therapeutic index and it can be expressed as the ratio LD₅₀/ED₅₀. Compositions which exhibit high therapeutic indices are preferred. While anti-CD3 antibody compositions that exhibit toxic side effects may be used, care should be taken to design a delivery system that targets such compounds to the site of affected tissue in order to minimize potential damage and, thereby, reduce side effects.

The data obtained from the cell cultures (e. g., of cells taken from an animal after mucosal administration of an anti-CD3 antibody) and animal studies can be used in formulating a range of dosage levels for use in humans. The dosage of anti-CD3 antibody compositions lies preferably within a range of mucosally available concentrations that include the ED₅₀ with little or no toxicity. The dosage may vary within this range depending upon the dosage form employed and the route of administration utilized. For any oral or mucosal anti-CD3 antibody compositions used in the methods described herein, the therapeutically effective dose can be estimated initially from assays of cell cultures (e. g., of cells taken from an animal after mucosal administration of an anti-CD3 antibody). A dose also may be formulated in animal studies based on efficacy in suitable animal models. Such information can be used to more accurately determine useful doses in humans.

As defined herein, a therapeutically effective amount of an anti-CD3 antibody (i.e., an effective dosage) depends on the antibody selected, the mode of delivery, and the condition to be treated. For instance, single dose amounts in the range of approximately 1 μg/kg to 1000 μg/kg may be administered; in some embodiments, about 5, 10, 50, 100, or 500 μg/kg may be administered. In some embodiments, e. g., pediatric subjects, about 1-100 μg/kg, e. g., about 25 or 50 μg/kg, of anti-CD3 antibody can be administered. The anti-CD3 antibody compositions can be administered from one or more times per day to one or more times per week, including for example once every day. The oral or mucosal anti-CD3 antibody compositions can be administered, e. g., for about 10 to 14 days or longer. The skilled artisan will appreciate that certain factors may influence the dosage and timing required to effectively treat a subject, including but not limited to the severity of the disease or disorder, type of disease or disorder, previous treatments, the general health and/or age of the subject, other diseases present, and persistence of the therapeutic effect.

Moreover, treatment of a subject with a therapeutically effective amount of the compounds may optionally include a single treatment or may optionally include a series of treatments.

The oral or mucosal anti-CD3 antibody compositions can also include one or more therapeutic agents useful for treating hepatitis. Such therapeutic agents can include, e. g., other antibodies, anti-viral agents or other anti-infectious agents suitable for treating infections described herein, such as interferon alfa-2b; and any agent suitable for treatment of diabetes, including but not limited to insulin, sulfonylureas (e. g., meglitinides and nateglinides), biguanides, thiazolidinediones, and alpha-glucosidase inhibitors, inter alia, as well as modification of diet or exercise regime.

The pharmaceutical compositions can be included in a container, pack, or dispenser together with instructions for administration.

Methods of Treatment

According to various embodiments of the present invention, the oral and mucosal anti-CD3 antibody compositions described herein can be administered to a subject to treat (which as described previously also includes preventing progression and/or delaying development of) disorders associated with hepatitis, including but not limited to infectious agents (including but not limited to viruses such as hepatitis A, B, C, D and E; other herpes viruses such as herpes simplex (HSV); cytomegalovirus (CMV); Epstein-Barr virus; other viruses such as yellow fever virus; HIV (human immunodeficiency virus), and adenoviruses; and non-viral infectious agents such as toxoplasma, Leptospira, Q fever and Rocky Mountain spotted fever; or any infectious agent which may cause hepatitis); toxins (including any toxic substance or any substance which is toxic with excessive intake, such as alcohol or medicines, for example due to any drug associated liver injury (DILI), including acetaminophen or any other drug that leads to liver damage); non-alcoholic steatohepatitis, or NASH, which is caused by obesity or diabetes, or a combination of these two conditions; liver disease associated with inflammatory bowel disease; hyperlipidemia, whether as the primary or only cause, or in association with NASH; and as a consequence of vascular disorders; or hepatitis of other etiology.

In some embodiments, the methods include administering an oral or mucosal anti-CD3 composition sufficient to produce an improvement in one or more clinical markers of hepatitis; for example, reduction or amelioration, or at least a reduction or absence of progression, of cirrhosis and/or fibrosis of the liver.

Cytokine Release Syndrome (CRS), which has been observed following parenteral administration of anti-CD3 antibodies, is not expected to be associated with oral administration of anti-CD3 antibodies, but the methods can include monitoring the subjects for signs and symptoms of CRS, particularly after the first few doses but also after a treatment hiatus with resumption of therapy; such methods are particularly useful in determining the safety of oral or mucosal administration of the anti-CD3 antibodies. CRS is associated with arthralgias, myalgias, fevers, chills, hypoxia, nausea, and vomiting; severe CRS can cause pulmonary edema and suffocation. In some embodiments, the methods include lowering the subject's temperature to less than about 37.8° C. (100° F.) before the administration of any dose of the anti-CD3 antibody compositions.

In some embodiments, the methods include screening the subject for clinical evidence of volume overload, uncontrolled hypertension, or uncompensated heart failure. In some embodiments, the methods include not administering the oral or mucosal anti-CD3 antibodies to subjects who have evidence of any of, volume overload, uncontrolled hypertension, or uncompensated heart failure. In some embodiments, the methods involve evaluating the subject's pulmonary function, and not administering the anti-CD3 antibodies to subjects who do not have a clear chest X-ray. In some embodiments, the methods include monitoring CD3+ T cell clearance and/or plasma levels of anti-CD3 antibody, and adjusting the dosage of the oral or mucosal anti-CD3 compositions accordingly.

In some embodiments, the methods include administering to the subject methylprednisolone sodium succinate 8.0 mg/kg, e. g., intravenously, e. g., 1-4 hours before administration of the oral or mucosal anti-CD3 antibody compositions. In some embodiments, the methods can include administering to the subject an anti-inflammatory agent, e. g., acetaminophen or antihistamine, before, concomitantly with, or after administration of the oral or mucosal anti-CD3 compositions.

In some embodiments, the methods include evaluating and/or monitoring a subject for anti-anti-CD3 antibodies, and discontinuing administration of the oral or mucosal anti-CD3 antibody compositions if the subject has anti-anti-CD3 antibody titers of greater than about 1:1000.

In some embodiments, the oral or mucosal anti-CD3 antibody compositions are administered concurrently with one or more second therapeutic modalities as described herein.

In some embodiments, the above treatment method may also optionally encompass monitoring liver function of the subject, before, during and/or after treatment.

Liver function may optionally be assessed according to any known assay or test, including but not limited to a blood test (including but not limited to a test to assay one or more of alanine aminotransferase (ALT), aspartate aminotransferase (AST) or gamma-glutamyl transpeptidase (GGT) and/or any ratios thereof) and/or a liver biopsy (for example optionally as a needle biopsy).

In some embodiments the subject optionally does not have an autoimmune disease and/or optionally does not have diabetes.

### EXAMPLES

Some embodiments of the present invention are further described in the following examples, which do not limit the scope of the invention described in the claims.

### Example 1

### Treatment of NASH—Preclinical Evaluation

The efficacy of oral anti-CD3 immune molecule, in this non-limiting example an anti-CD3 antibody (aCD3), was demonstrated in ob/ob mice, which were found to have reduced steatohepatitis (fatty livers) after administration of an anti-CD3 antibody (Yaron Ilan et al; “Induction of regulatory T cells decreases adipose inflammation and alleviates insulin resistance in ob/ob mice”; PNAS, 2010 May 25;107(21):9765-70, electronic publication on May 5 2010).

Methods

Mice. C57BL/6 (B6), ob/ob, or CD1d−/− mice, age 8-10 weeks, were purchased from Jackson Laboratory (Bar Harbor, Me., USA). Mice were housed in a pathogen-free animal facility

Antibodies and reagents. Hamster anti-mouse CD3 antibody (clone 145-2C11) and Rat anti-TGF-β was purchased from BIO X CELL (West Lebanon, N.H.) and control hamster IgG was purchased from Jackson ImmunoResearch Laboratories, PA, USA. Anti CD3 (clone 145-2C11) for in vitro stimulation and reagents for FACS staining were purchased from BD PharMingen, CA, USA: CD16/CD32 (FcBlock); FITC, PE, or APCconjugated anti CD4 (L3T4); and PE-conjugated anti CD25 (PC61). Affinity-purified biotinylated goat anti-LAP polyclonal antibody and Strep-Avidin APC was purchased from R&D Systems, MN, USA. 7-AAD for staining dead cells was purchased from Sigma-Aldrich, MO, USA.

Oral administration and injections. Mice were fed a total volume of 0.2 ml by gastric intubation with an 18-gauge stainless steel feeding needle (Thomas Scientific, NJ, USA). Mice were fed once a day for five consecutive days with either phosphate buffered saline (PBS), hamster isotype control (IC, 5mg/feeding), or anti-CD3 antibody (5mg/feeding), dissolved in ethanol and emulsified in PBS.

Histology. The liver, pancreas, and muscle were removed from control or treated mice and placed in 4% formalin followed by paraffin embedding. Five sections were prepared from each organ. The tissues were stained for Hematoxylin eosin and liver sections were additionally stained with oil-red-o. All sections were blindly scored by a pathologist.

Analysis of adipose tissue, liver enzymes, cholesterol and blood glucose. Mice (4/group) were fed PBS or anti-CD3 (5 μg) for 5 consecutive days. 72 h after the last feeding perigonadal white fat was collected and fat paraffin sections were stained with H&E. Pictures were taken at x 40 magnification. Also at 72 h after the last feeding (6 mice/group) white fat near or surrounding mesenteric lymph nodes was used to isolate adipocytes. Adipocytes were stained with fluorescent antibodies to CD11b and F4/80 or CD4 then fixed and permeablized and stained with antibody to Foxp3. RNA of adipocytes isolated from perigonadal fats were used in RTPCR for cytokine expression of IL-10, TNF-α and TGF-β. CD4+ T cells were negatively selected from spleens of PBS or anti-CD3 fed mice and co-cultured with adipocytes from control mice at 1:1 ratio for 5 days. CD4+ T cells were eliminated from co-culture by positive selection leaving adipocytes for extraction of RNA used in RTPCR for cytokine expression. Liver enzymes AST and ALT and cholesterol were measured by the Clinical Biochemistry Lab at Brigham and Women's hospital using a Seralyzer system in a bind-folded fashion. Blood glucose level was measured using Diastix reagent strips according to manufacturer's protocol (Fisher Scientific).

Statistical analysis. Statistical significance was assessed by the two-tailed Student's t-test.

When there were more than two groups compared, differences were analyzed using one-way ANOVA. P-values<0.05 were considered significant.

### Results

Oral anti-CD3 decreases glucose, liver enzymes and cholesterol in ob/ob mice. Ob/ob mice were fed daily with 5 μg anti-CD3 for five consecutive days and blood glucose, liver enzymes and lipid levels were measured ten days post feeding. The doses studied were based on previous studies of anti-CD3 in animal models (data not shown). As shown in Table 1, a decrease in blood glucose in anti-CD3 treated animals (316 mg %) was observed compared to control animals fed PBS (367 mg %).

TABLE 1
Oral Anti-CD3 decreases levels of glucose
and liver enzymes in ob/ob mice.

| | PBS | Anti-CD3 |
|---|---|---|
| Glucose (mg %) | 367 + 62 | 316 + 48 |
| AST (U/l) | 416 + 58 | 296 + 44 |
| Cholesterol (mg %) | 218 + 39 | 219 + 37 |

Ob/ob mice (5/group) were fed daily with 5 μg anti-CD3 daily for five days and blood glucose, liver enzymes and lipid levels were measured ten days post feeding.

A decrease in levels of serum aspartate aminotransferase (AST) was observed in animals fed anti-CD3 (296U/l) compared to control animals (416U/l). Serum cholesterol levels were lower in mice fed anti-CD3 (219 mg %) vs. PBS (218 mg %). No change in the weight of animals was observed in the anti-CD3 group compared to controls. No effect was observed when an isotype control antibody for anti-CD3 was given as previously described.

Oral anti-CD3 reduces hepatic fat accumulation and pancreatic hyperplasia. After observing these metabolic effects, the effect of oral anti-CD3 was measured in ob/ob mice by pathologic analysis of pancreas, liver and muscle. The results demonstrated a reduction in pancreatic hyperplasia and hepatic fat accumulation.

### Example 2

### Treatment of NASH—Clinical Trial

The safety, efficacy and immune modulation of anti-CD3 oral immune molecule therapy (aCD3) was assessed in a clinical trial of patients with Non-Alcoholic Steatohepatitis (NASH) and altered glucose metabolism. NASH patients received daily doses of aCD3 MAb over a 1-month time period, at dosage levels of 0 (placebo group) and 0.2, 1.0 or 5.0 mg. It is noted that other dosing intervals (longer than 1 month) and frequencies (e.g., semi-weekly, weekly) and dosage levels may be useful for treatment or preventing progression and/or delaying development of NASH.

Methods.

Safety of aCD3 was assessed by monitoring the subjects for reported adverse events (AEs) and by interpreting the results of the various laboratory tests for safety, which included general blood chemistry, liver and kidney functions, levels of immune safety markers including CD3, CD4 and CD8, and complete blood count (CBC) including white blood cells (WBC) differential, as well as by comparing the frequency and patterns of AEs in the aCD3 treatment groups to those of the placebo group. Immune-modulatory changes were monitored by changes in levels of cytokines secreted by T cells and by levels of cell surface markers for Treg and other immune system markers. Efficacy was based on: one or more efficacy biomarkers, each of which is known to deviate from normal in patients with NASH, and which included the following: glucose tolerance test (GTT), Homeostatic Model Assessment (HOMA score), alanine aminotransferase (ALT), aspartate aminotransferase (AST), gamma-glutamyl transpeptidase (GGT), total cholesterol, low density lipoprotein (LDL), and triglycerides. It should be noted that all of these immune-modulation and efficacy evaluations, other than liver biopsy which is done by needle biopsy, are readily performed using established and available technologies on blood samples from patients during and after the course of aCD3 therapy.

The assessments for immune modulation and efficacy were ascertained for each subject by comparing values in efficacy parameters before aCD3 therapy to those during and after aCD3 therapy, as well as by comparing overall changes in immune-modulation and efficacy parameters among one or more of the three aCD3 treatment groups compared to the placebo group. Statistical evaluations were performed by group analysis comparing means for each treatment group vs. placebo group by t-test as well as by individual analysis comparing the number of subjects per treatment group with >10% increased improvement in the particular parameter vs. the same for the placebo group by t-test.

Results

Safety: The immunotherapy was found to be very safe and well tolerated, with no drug-related adverse effects or systemic complaints in any of the three dosage groups, as measured by blood hematology, chemistry, and general physical signs. During the course of immunotherapy and compared to placebo, there were no changes in blood levels of CD3-positive cells or of CD4-positive or CD8-positive cells which are indicators of immunological safety. Some subjects had increased levels of serum antibodies directed against the MAb. The safety data are consistent with the Phase 1 safety data for oral anti-CD3 immunotherapy in healthy subjects.

Efficacy biomarkers: The immunotherapy resulted in positive trends in clinical parameters in groups receiving oral anti-CD3 but not in the placebo group—some of these trends were statistically significant in spite of the very small group sizes. These positive trends on efficacy were reduced blood levels of two liver enzymes (ALT, AST), and improved glucose metabolism in the GTT, which are favorable outcomes for subjects with NASH or metabolic syndrome and for subjects with type-2 diabetes or altered glucose metabolism. Several of the positive efficacy trends persisted to Day 60 following cessation of immunotherapy at Day 30.

Immune modulation markers: The immunotherapy induced LAP+ regulatory T cells, which generally persisted to Day 60 in some of the patients. Subjects in groups receiving the MAb showed increases in such markers, while those receiving placebo did not show such increases. Other immunomodulatory effects included trends in the induction of cytokines, which are natural molecules that influence the immune system, in particular for TGFβ which has been shown in preclinical studies to be required for the induction of LAP+ Tregs in oral anti-CD3 immunotherapy.

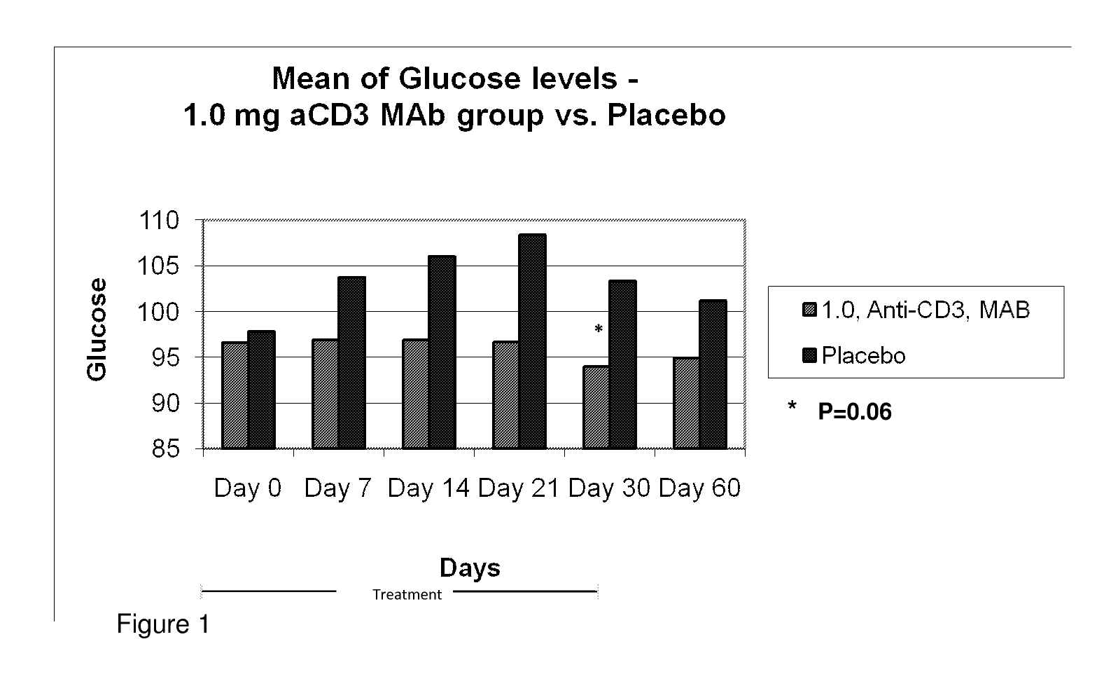

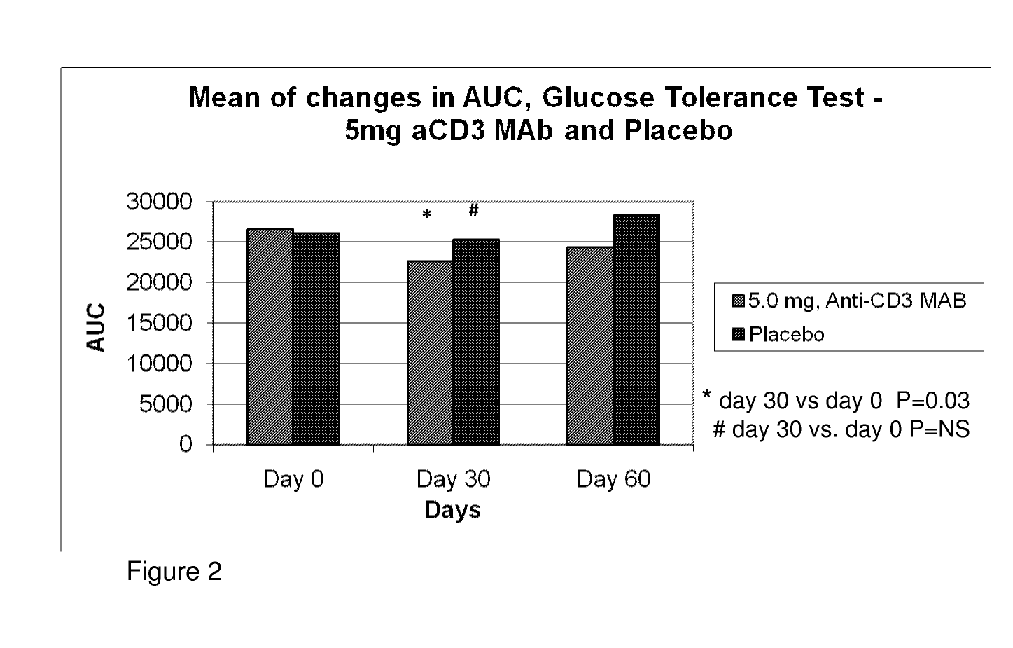

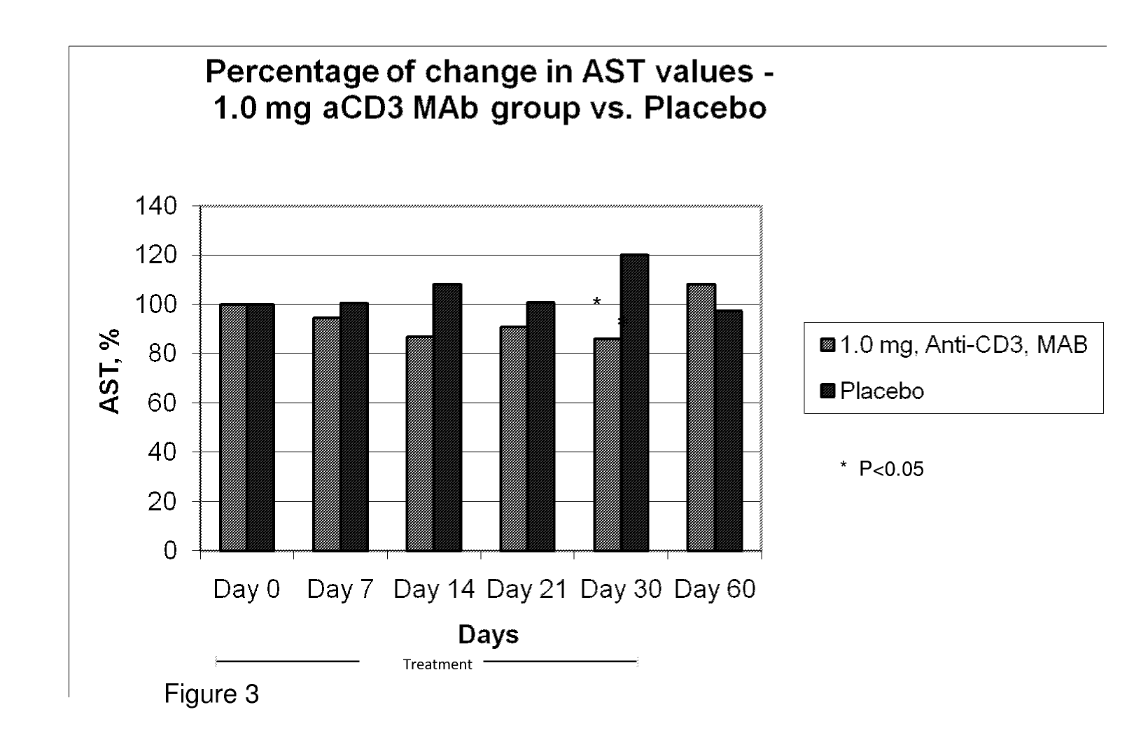

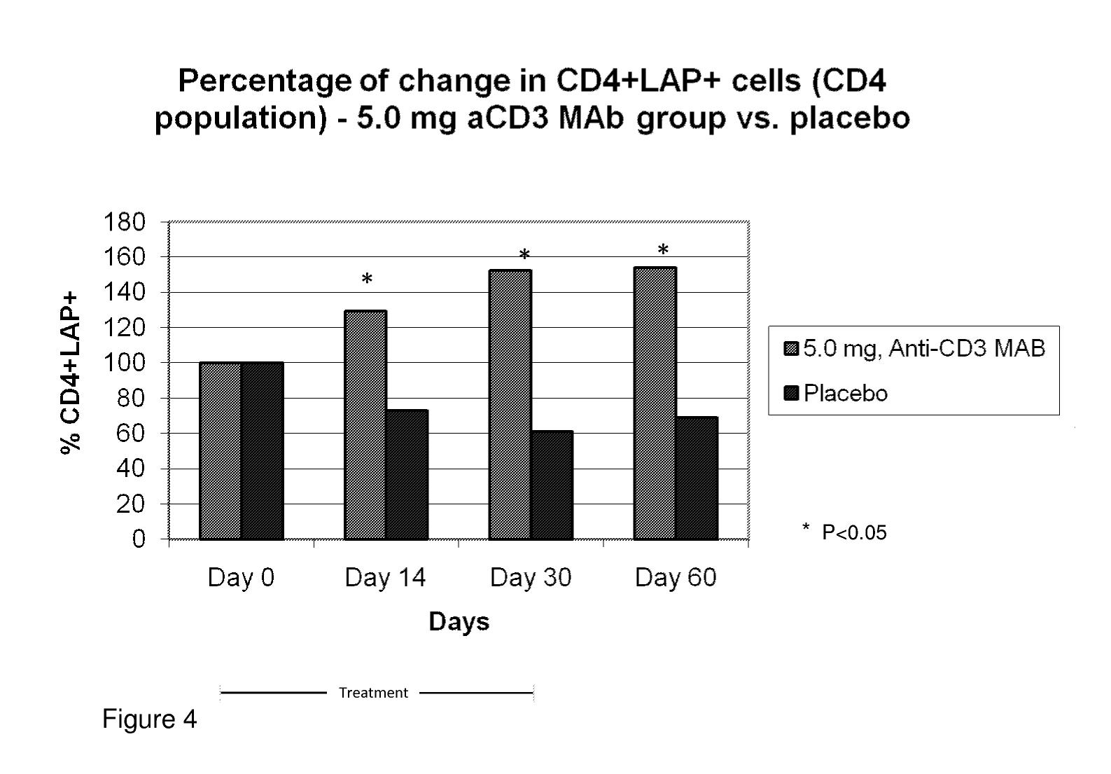

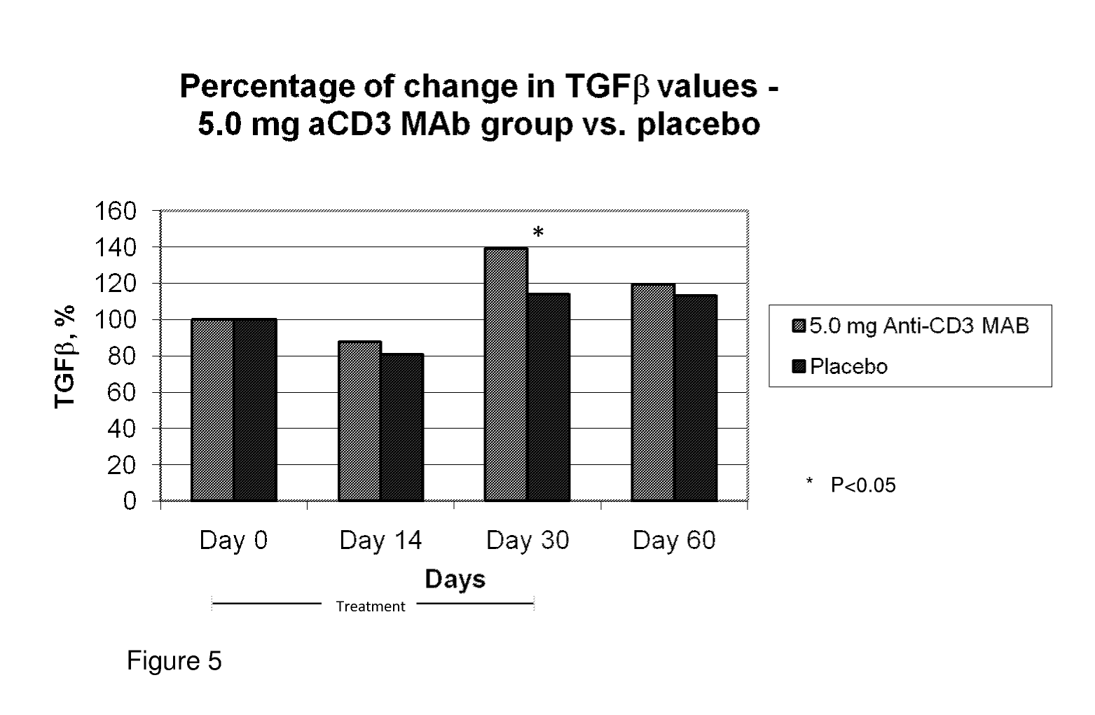

Examples of improvements in efficacy biomarkers and immune nodulation are shown in FIGS. 1-5, which relate to changes from pre-treatment (Day 0) to post-treatment (Day 30) timepoints in patients. FIG. 1 relates to mean blood glucose levels, which are lower (and hence more controlled) in patients receiving the immunotherapy. FIG. 2 relates to the mean of changes for the AUC (area under the curve) for a glucose tolerance test; again better results are found in patients receiving the immunotherapy. FIG. 3 relates to AST levels; again better results are found in patients receiving the immunotherapy. FIG. 4 relates to the percentage of change in levels of the CD4³⁰ LAP⁺ population, while FIG. 5 relates to the percentage of change in TGFβ levels; again better results are found in patients receiving the immunotherapy.

### Example 3

### Treatment of Hepatitis C

The efficacy of aCD3 is assessed in a clinical trial of patients with chronic liver infection caused by hepatitis C virus (HCV). Chronic HCV patients may be taking interferon (IFn) therapy, which is a licensed product for chronic HCV. Subjects are taking IFn continually, or alternatively have failed IFn therapy, either for reasons of lack of efficacy or poor tolerability leading to withdrawal from IFn therapy. A clinical trial is performed either in chronic HCV patients receiving IFn therapy or withdrawn from IFn therapy. Chronic HCV patients receive doses of aCD3 over a period, for example a 1- to 6-month interval or longer at a dosing frequency of, for example, daily to weekly at dosage levels of, for example, 0 (placebo group), 0.2, 1.0 or 5.0 mg. It is noted that other dosing intervals and frequencies and dosage levels may be useful for treatment or preventing progression and/or delaying development of the disease or condition. Safety of aCD3 is assessed by monitoring the subjects for reported adverse events (AEs) and by interpreting the results of the various laboratory tests for safety which may include general blood chemistry, liver and kidney functions, and CBC including WBC differentials, as well as by comparing the frequency and patterns of AEs in the aCD3 treatment groups to that of the placebo group. Efficacy is based on improvement in one or more of the following parameters, each of which is known to deviate from normal in patients with chronic HCV: levels of HCV ribonucleic acid (RNA), ALT, AST, GGT, and liver biopsy. It should be noted that all of these efficacy evaluations, other than liver biopsy which is done by needle biopsy, are readily performed on blood samples from patients during and after the course of aCD3 therapy. The assessments for efficacy are ascertained for each subject by comparing values in efficacy parameters before aCD3 therapy to those during and after aCD3 therapy, as well as by comparing overall changes in efficacy parameters among one or more of the three aCD3 treatment groups compared to the placebo group.

### Other Embodiments

It is to be understood that while the invention has been described in conjunction with the detailed description thereof, the foregoing description is intended to illustrate and not limit the scope of the invention, which is defined by the scope of the appended claims. Optionally any one or more embodiments, sub-embodiments and/or components of any embodiment may be combined. Other aspects, advantages, and modifications are within the scope of the following claims.

1. A method of treating or preventing progression of hepatitis in a subject, comprising administering to the subject an anti-CD3 immune molecule orally or muco sally.

2. Use of an anti-CD3 immune molecule for oral or mucosal administration to a subject to treat or prevent progression of hepatitis.

3. A pharmaceutical composition comprising an anti-CD3 immune molecule suitable for oral or mucosal administration, in a dosage suitable for treatment or preventing progression of hepatitis.

4. The method of claim 1, wherein the hepatitis comprises inflammation of the liver.

5. The method of claim 4, wherein said hepatitis has a cause selected from the group consisting of an infectious agent; toxins; any liver disease associated with or exacerbated by obesity or diabetes; liver disease associated with inflammatory bowel disease; liver disease associated with a vascular disorder.

6. The method of claim 5, wherein said hepatitis has a cause selected from the group consisting of a viral or non-viral infectious agent.

7. The method of claim 6, wherein said hepatitis has a cause selected from the group consisting of hepatitis A, B, C, D and E; herpes viruses; cytomegalovirus; Epstein-Barr virus; yellow fever virus, HIV (human immunodeficiency virus), and adenoviruses.

8. The method of claim 7, wherein said hepatitis is caused by viral hepatitis C.

9. The method of claim 6, wherein said hepatitis has a cause selected from the group consisting of toxoplasma, Leptospira, Q fever and Rocky Mountain spotted fever.

10. The method of claim 6, wherein said hepatitis has a cause selected from the group consisting of alcohol or medicines, or is associated with any drug associated liver injury (DILI).

11. The method of claim 6, wherein said hepatitis has a cause associated with obesity or diabetes, or a combination of these two conditions.

12. The method of claim 11, wherein said hepatitis is caused by one or more of non-alcoholic steatohepatitis (NASH), or hyperlipidemia, whether as the primary or only cause, or in association with NASH.

13. The method of claim 12, wherein said hepatitis is caused by NASH.

14. The method of claim 13, wherein treatment with said anti-CD3 immune molecule ameliorates a NASH-associated parameter measured according to an assay selected from the group consisting of glucose tolerance test (GTT), Homeostatic Model Assessment (HOMA score), alanine aminotransferase (ALT) level, aspartate aminotransferase (AST) level, gamma-glutamyl transpeptidase (GGT) level, total cholesterol level, low density lipoprotein (LDL) level or ratio with HDL (high density lipoprotein), triglyceride level and steatohepatitis as assessed through liver biopsy.

15. The method of claim 1, wherein oral or mucosal administration comprises one or more of pulmonary, buccal, nasal, intranasal, sublingual, rectal, or vaginal administration.

16. The method of claim 1, wherein said anti-CD3 immune molecule comprises an anti-CD3 antibody.

17. The method of claim 16, wherein said anti-CD3 antibody comprises a molecule selected from the group consisting of a whole antibody or active fragments thereof.

18. The method of claim 17, wherein the anti-CD3 antibody is selected from the group consisting of a murine mAb, a humanized mAb, a human mAb, and a chimeric mAb.
